(19) United States
(12) Patent Application Publication
(10) Pub. No.: US 2023/0023687 A1
(43) Pub. Date: Jan. 26, 2023

(54) ALICYCLIC ACRYLATE COMPOUND, ALICYCLIC EPOXY ACRYLATE COMPOUND, CURABLE COMPOSITION, AND CURED PRODUCT

(71) Applicant: ENEOS Corporation, Tokyo (JP)

(72) Inventors: Kenta UE, Tokyo (JP); Takeshi KOIKE, Tokyo (JP); Atsushi KAMEYAMA, Tokyo (JP)

(73) Assignee: ENEOS Corporation, Tokyo (JP)

(21) Appl. No.: 17/782,082

(22) PCT Filed: Dec. 1, 2020
(86) PCT No.: PCT/JP2020/044604

§ 371 Date: Jun. 2, 2022

(30) Foreign Application Priority Data
Dec. 2, 2019 (JP) 2019-218298

(51) Int. Cl. C08F 222/10; C08F 220/32; C08F 220/06; C08F 220/14; C08F 2/50; C08K 5/09

(52) U.S. Cl. CPC C08F 222/1067; C08F 220/32; C08F 220/06; C08F 220/14; C08F 2/50; C08K 5/09

(57) ABSTRACT

The present invention provides an alicyclic epoxy acrylate compound that can have high heat resistance in the form of a cured product when contained in a curable composition, a curable composition, and a cured product.

An alicyclic epoxy acrylate compound represented by the following Formula (1) where A denotes an oxygen atom and a curable composition comprising thereof are used:

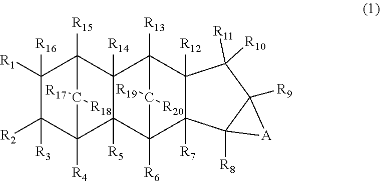

wherein one of R₁ and R₂ is a (meth)acryloyloxy group, the other of R₁ and R₂ is a hydrogen atom, R₃ to R₂₀ are each independently selected from the group consisting of a hydrogen atom, an alkyl group, and an alkoxy group, and A is an oxygen atom or A is not present, and a carbon atom to which R₈ binds and a carbon atom to which R₉ binds together form a double bond.

### CROSS-REFERENCE TO RELATED APPLICATIONS

The present patent application is based upon and claims the benefit of priority from Japanese Patent Application No. 2019-218298 (filed on Dec. 2, 2019). The entire disclosures of the above described patent applications are incorporated herein by reference.

### BACKGROUND OF THE INVENTION

### Field of the Invention

The present invention relates to an alicyclic acrylate compound, an alicyclic epoxy acrylate compound, a curable composition, and a cured product.

### Background Art

An epoxy group-containing (meth)acrylic compound and an acrylate having an alicyclic skeleton are generally a compound that is useful in the fields of resists, sealing materials, coatings, inks, adhesives, sealants, and the like. Here, an acrylate containing an epoxy group with an alicyclic skeleton is known to have properties suitable for outdoor use. They generally exert a certain performance by the ring opening reaction of the oxirane ring present in the structure, the reaction of the ethylene-based unsaturated group of the acrylate, or the like.

For example, Patent Document 1 discloses that a vinyl group-containing alicyclic acrylate compound having a specific alicyclic skeleton is particularly useful for coating.

Further, Patent Document 2 discloses, as a resin composition containing an epoxy group-containing (meth)acrylic compound, a cured product of a resin composition containing a specific epoxy group-containing (meth)acrylate that is excellent in heat resistance and the like.

However, conventional epoxy group-containing (meth)acrylic compounds as suggested in Patent Documents 1 and 2 had room for further improvement in terms of heat resistance of cured products.

### PRIOR ART DOCUMENTS

### Patent Documents

Patent Document 1: JP H11-209326 A
Patent Document 2: JP 2006-131866 A

### SUMMARY OF THE INVENTION

### Problems to be Solved by the Invention

In applications such as resists, sealing materials, and coatings, there is still a cured product having further improved high heat resistance such that it can be adapted to more severe use conditions.

Therefore, an object of the present invention is to provide an alicyclic epoxy acrylate compound that can have high heat resistance in the form of a cured product when contained in a curable composition. Another object of the present invention is to provide an epoxy group-containing (meth)acrylic polymer and a radical-polymerizable compound, which can have high heat resistance in the form of a cured product when contained in a curable composition. Yet another object of the present invention is to provide a curable composition for obtaining a cured product having high heat resistance.

### Means for Solving the Problems

[1] An alicyclic acrylate compound represented by the following Formula (1):

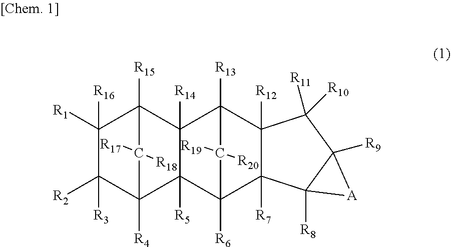

wherein one of R₁ and R₂ is a (meth)acryloyloxy group,

the other of R₁ and R₂ is a hydrogen atom,

R₃ to R₂₀ are each independently selected from the group consisting of a hydrogen atom, an alkyl group, and an alkoxy group, and

A is an oxygen atom or A is not present, and a carbon atom to which R₈ binds and a carbon atom to which R₉ binds together form a double bond.
[2] An alicyclic acrylate-type polymerizable monomer comprising the alicyclic acrylate compound according to [1].
[3] The alicyclic acrylate compound according to [1], which is an alicyclic epoxy acrylate compound represented by the Formula (1), wherein A is an oxygen atom.
[4] An alicyclic epoxy acrylate-type polymerizable monomer comprising the alicyclic epoxy acrylate compound according to [3].
[5] A curable composition comprising at least the alicyclic epoxy acrylate-type polymerizable monomer compound according to [4].
[6] The curable composition according to [5], which further comprises at least one selected from the group consisting of a curing agent, a thermal cationic polymerization initiator, and a photo-cationic polymerization initiator.
[7] The curable composition according to [6], wherein the curing agent is an acid anhydride-based compound.
[8] The curable composition according to any one of [5] to [7], which further comprises a thermal radical polymerization initiator.
[9] The curable composition according to any one of [5] to [8], which further comprises an epoxy compound that is different from the alicyclic epoxy acrylate compound.
[10] The curable composition according to any one of [5] to [9], which further comprises a radical-polymerizable compound that is different from the alicyclic epoxy acrylate compound.
[11] An epoxy group-containing (meth)acrylic polymer comprising at least a polymerization unit represented by the following Formula (2):

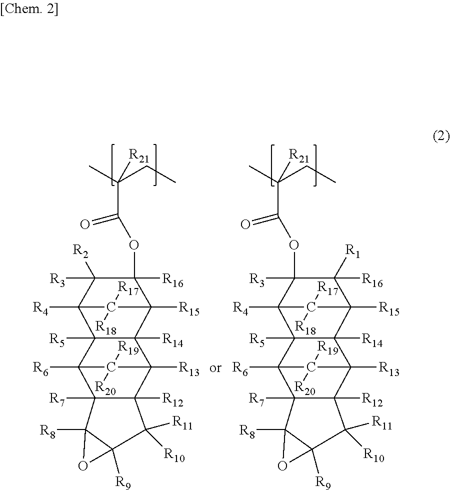

wherein R₁ and R₂ are each a hydrogen atom,

R₃ to R₂₀ are each independently selected from the group consisting of a hydrogen atom, an alkyl group, and an alkoxy group, and

R₂₁ is a hydrogen atom or a methyl group.
[12] A curable composition comprising at least the epoxy group-containing (meth)acrylic polymer according to [11].
[13] The curable composition according to [12], which further comprises a photo-cationic polymerization initiator.
[14] The curable composition according to [12] or [13], which further comprises an epoxy compound that is different from the epoxy group-containing (meth)acrylic polymer.
[15] A radical-polymerizable compound comprising at least a polymerization unit represented by the following Formula (3):

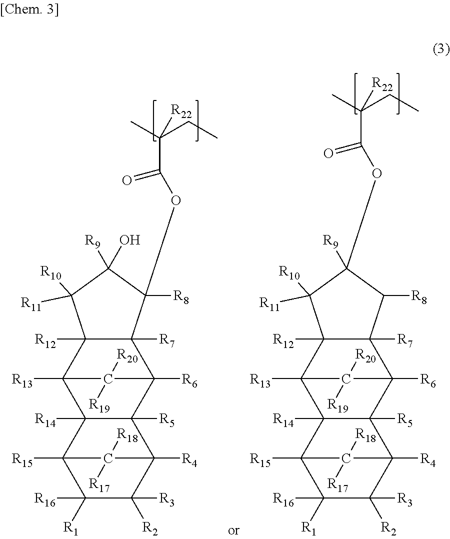

wherein one of R₁ and R₂ is a (meth)acryloyloxy group,

the other of R₁ and R₂ is a hydrogen atom,

R₃ to R₂₀ are each independently selected from the group consisting of a hydrogen atom, an alkyl group, and an alkoxy group, and

R₂₂ is selected from a hydrogen atom and a methyl group.
[16] The radical-polymerizable compound according to [15], which further comprises a polymerization unit derived from at least one acrylic compound selected from (meth)acrylic acid ester and (meth)acrylic acid.
[17] A curable composition comprising at least the radical-polymerizable compound according to [15] or [16].
[18] The curable composition according to [17], which further comprises a photoradical polymerization initiator.
[19] The curable composition according to [17] or [18], which further comprises a radical-polymerizable compound that is different from the radical-polymerizable compound.
[20] A method of producing an epoxy group-containing acrylic polymer, comprising a step of radically polymerizing the alicyclic epoxy acrylate compound according to [3].
[21] A method of producing a radical-polymerizable compound, comprising a step of performing radical polymerization of a polymer of at least one acrylic compound selected from (meth)acrylic acid ester and (meth)acrylic acid with the alicyclic epoxy acrylate compound according to [3].

### Effect of the Invention

According to the present invention, an alicyclic epoxy acrylate compound that can have high heat resistance in the form of a cured product when contained in a curable composition can be provided. In addition, according to the present invention, an epoxy group-containing (meth)acrylic polymer and a radical-polymerizable compound, which can have high heat resistance in the form of a cured product when contained in a curable composition can be provided. Further, according to the present invention, a curable composition for obtaining a cured product having high heat resistance can be provided.

### BRIEF DESCRIPTION OF THE DRAWINGS

FIG. 1 shows an ¹H-NMR chart of the alicyclic acrylate-type polymerizable monomer synthesized in Preparation Example 1-1.

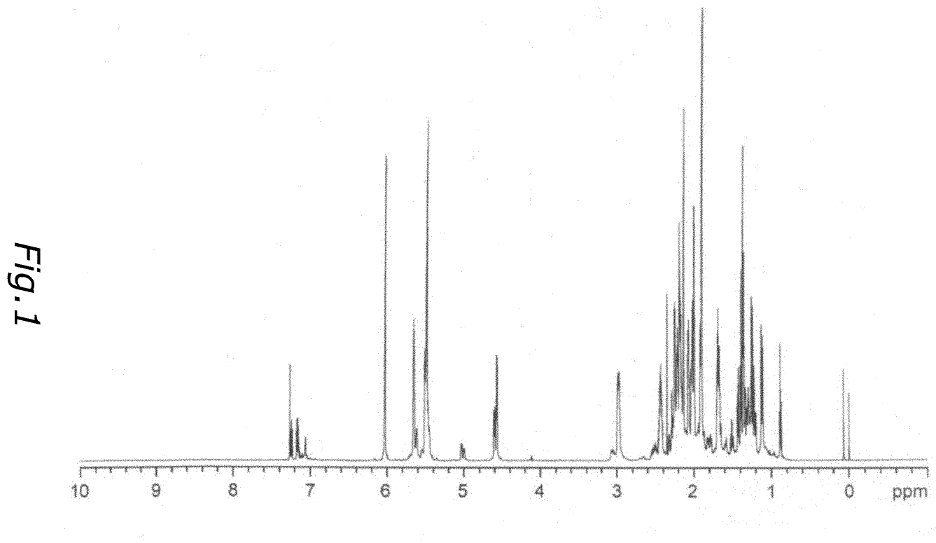

FIG. 2 shows a ¹³C-NMR chart of the alicyclic acrylate-type polymerizable monomer synthesized in Preparation Example 1-1.

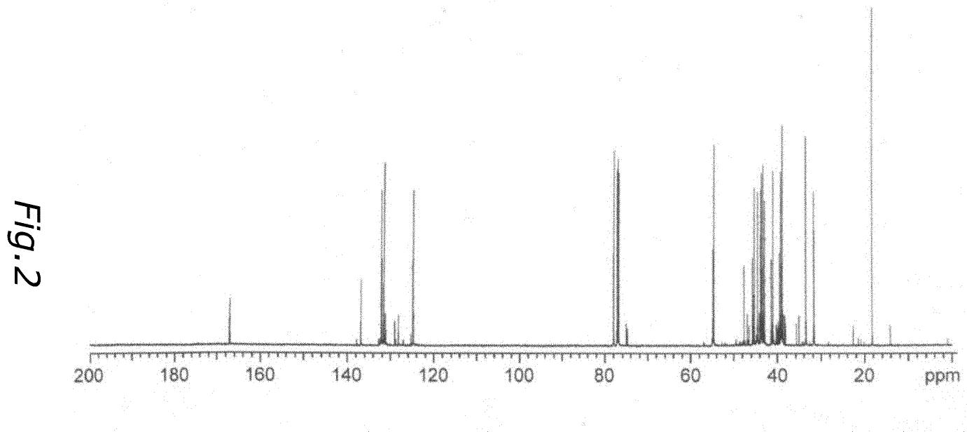

FIG. 3 shows an 1H-NMR chart of the alicyclic epoxy acrylate-type polymerizable monomer (A1) synthesized in Preparation Example 1-2.

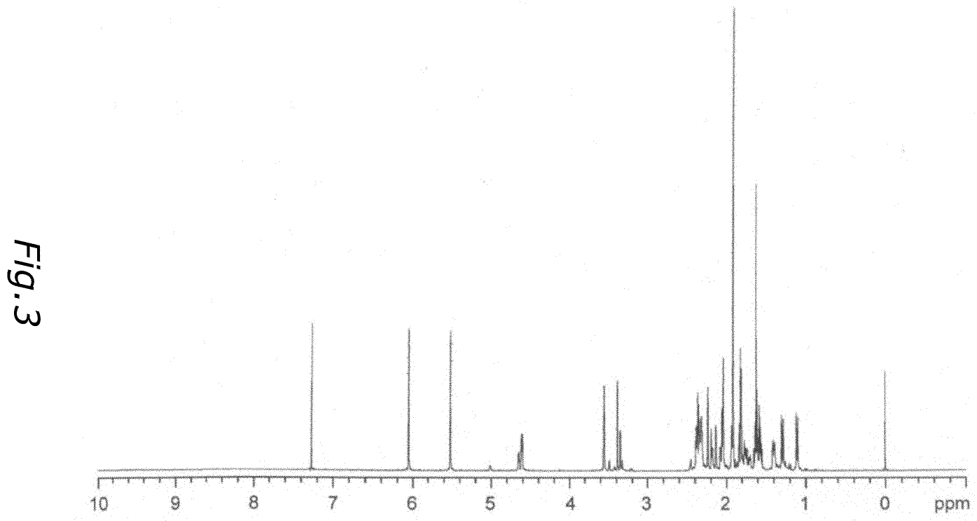

FIG. 4 shows a ¹³C-NMR chart of the alicyclic epoxy acrylate-type polymerizable monomer (A1) synthesized in Preparation Example 1-2.

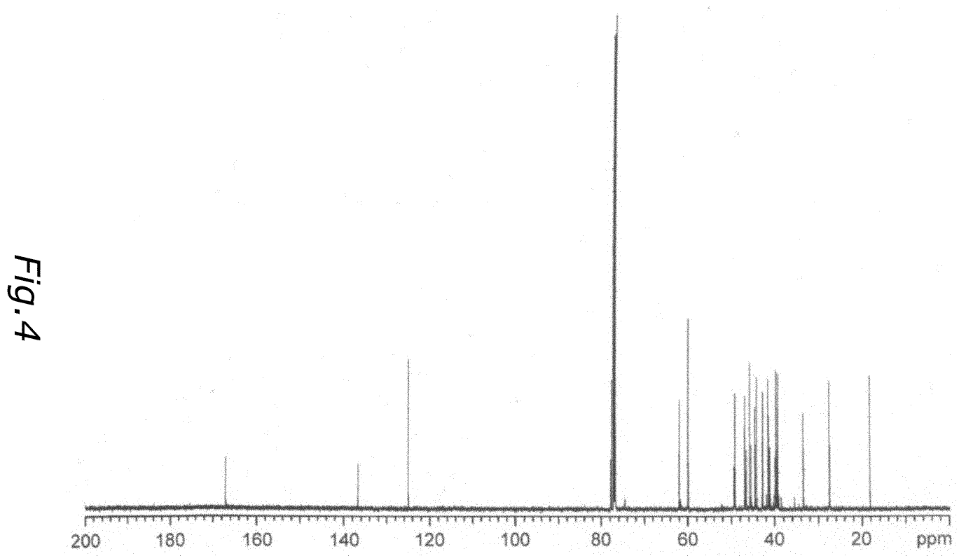

FIG. 5 shows an infrared absorption spectrum (IR) of the epoxy group-containing (meth)acrylic polymer obtained in Example 3-1.

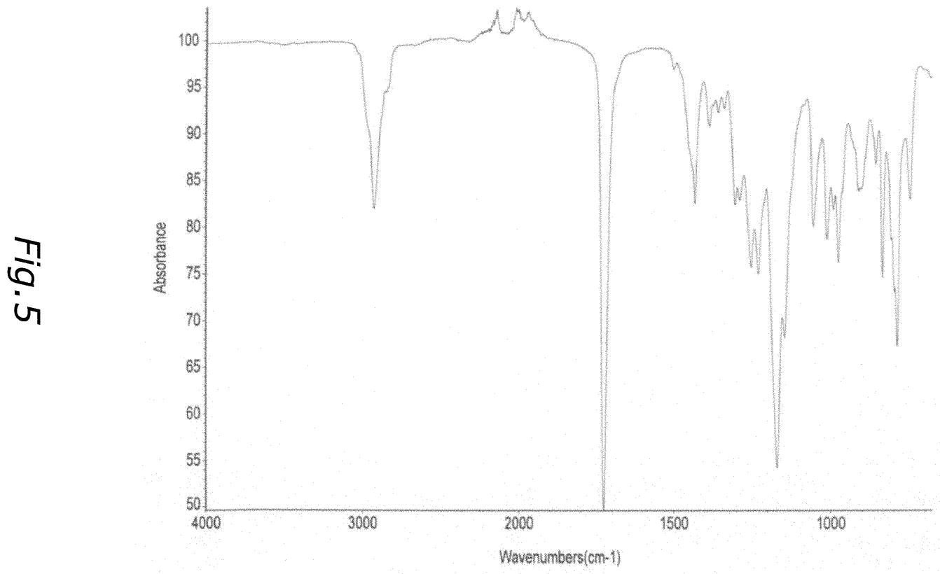

FIG. 6 shows an infrared absorption spectrum (IR) of the radical-polymerizable compound obtained in Example 4-1.

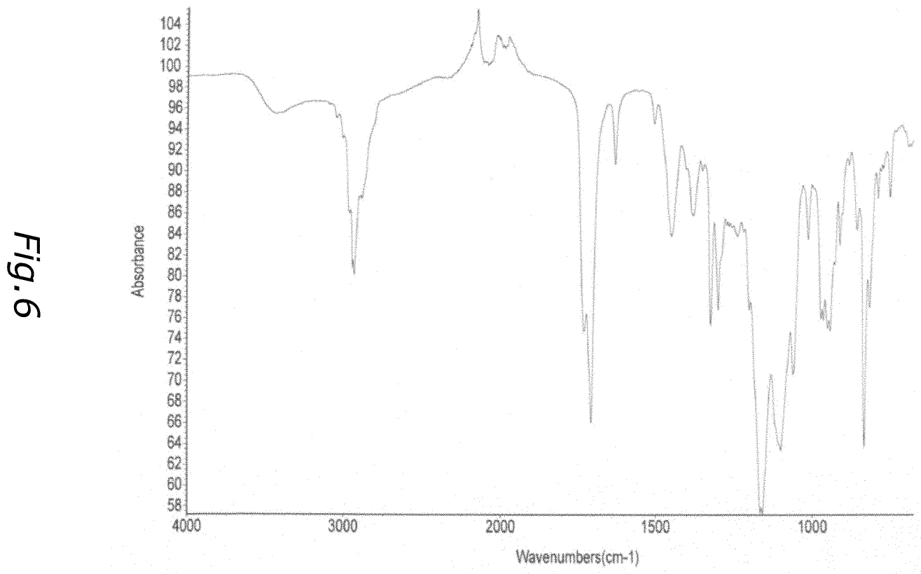

### DETAILED DESCRIPTION OF THE INVENTION

### Definitions

In the present specification, the terms “part(s),” “%” and the like used to describe the composition are represented on a mass basis, unless otherwise specified.

### Alicyclic Acrylate Compound

According to one embodiment of the present invention, the alicyclic acrylate compound is an alicyclic acrylate compound represented by the following Formula (1). A curable composition, which contains an alicyclic epoxy acrylate compound for which A is an oxygen atom in the following Formula (1) for the alicyclic acrylate compound, can be provided. In addition, an alicyclic acrylate compound of the following Formula (1) where A is not present and the carbon atom to which R₈ binds and the carbon atom to which R₉ binds together form a double bond can be used as a starting material for an alicyclic epoxy acrylate compound.

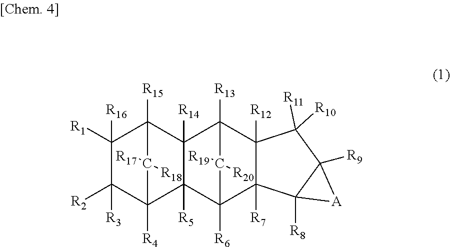

wherein, one of R₁ and R₂ is a (meth)acryloyloxy group,

the other of R₁ and R₂ is a hydrogen atom,

R₃ to R₂₀ are each independently selected from the group consisting of a hydrogen atom, an alkyl group, and an alkoxy group, and

A is an oxygen atom or A is not present, and a carbon atom to which R₈ binds and a carbon atom to which R₉ binds together form a double bond.

According to one embodiment of the present invention, an alicyclic acrylate-type polymerizable monomer is provided. Here, the alicyclic acrylate-type polymerizable monomer may be a single alicyclic acrylate compound or a mixture of alicyclic acrylate compounds. The polymerizable monomer may be, for example, one or more kinds of alicyclic acrylate compounds, a combination of two or more kinds of alicyclic acrylate compounds, or a combination of two kinds of alicyclic acrylate compounds.

In the alicyclic acrylate compound represented by the following Formula (1), one of R₁ and R₂ is a (meth)acryloyloxy group, the other of R₁ and R₂ is a hydrogen atom, preferably, one of R₁ and R₂ is a (meth)acryloyloxy group, and the other of R₁ and R₂ is a hydrogen.

According to another embodiment of the present invention, in the alicyclic acrylate compound represented by Formula (1), R₁ is a (meth)acryloyloxy group, R₂ is a hydrogen atom or R₁ is a hydrogen atom, R₂ is a (meth)acryloyloxy group, R₁ is preferably a methacryloyloxy group, R₂ is a hydrogen atom or R₁ is a hydrogen atom, and R₂ is a methacryloyloxy group.

In the alicyclic acrylate compound represented by the following Formula (1), R₃ to R₂₀ are each independently selected from the group consisting of a hydrogen atom, an alkyl group, and an alkoxy group, preferably selected from the group consisting of a hydrogen atom and an alkyl group, and more preferably a hydrogen atom.

In the alicyclic acrylate compound represented by Formula (1), preferably, R₃ and R₁₆ are each a hydrogen atom,

The number of carbon atoms in alkyl groups of R₃ to R₂₀ in the above-described Formula (1) is, but is not particularly limited to, preferably from 1 to 10 and more preferably from 1 to 5. The above-described alkyl groups may be a linear alkyl group or branched alkyl group.

Examples of the linear alkyl group include a methyl group, an ethyl group, an n-propyl group, and an n-butyl group.

Examples of the branched alkyl group include an isopropyl group, an isobutyl group, and a t-butyl group.

The number of carbon atoms in alkoxy groups of R₃ to R₂₀ in the above-described Formula (1) is, but is not particularly limited to, preferably from 1 to 10 and more preferably from 1 to 5.

Examples of an alkoxy group include a methoxy group, an ethoxy group, a propyloxy group, an i-propyloxy group, an n-butoxy group, an i-butoxy group, an s-butoxy group, and a t-butoxy group.

According to a preferred embodiment of the present invention, the alicyclic acrylate-type polymerizable monomer comprising the alicyclic acrylate compound represented by Formula (1) comprises a compound (1-i) represented by Formula (1) where R₁ is a (meth)acryloyloxy group and R₂ is a hydrogen atom, a compound (1-ii) represented by Formula (1) where R₂ is a (meth)acryloyloxy group and R₁ is a hydrogen atom, or a mixture thereof. Hereinafter, an alicyclic acrylate-type polymerizable monomer comprising an alicyclic (epoxy)acrylate compound represented by Formula (X) is referred to as an “(epoxy)acrylate-type polymerizable monomer represented by Formula (X).”

According to a preferred embodiment of the present invention, the alicyclic acrylate-type polymerizable monomer comprising the alicyclic acrylate compound represented by Formula (1) comprises a compound (1-i-1) represented by Formula (1) where R₁ is a (meth)acryloyloxy group and R₂ to R₂₀ are each a hydrogen atom, a compound (1-ii-1) represented by Formula (1) where R₂ is a (meth)acryloyloxy group and R₁ and R₃ to R₂₀ are each a hydrogen atom, or a mixture thereof.

According to one embodiment of the present invention, an alicyclic acrylate compound for which A is not present and the carbon atom to which R₈ binds and the carbon atom to which R₉ binds together form a double bond in Formula (1) above is represented by the following Formula (1-1).

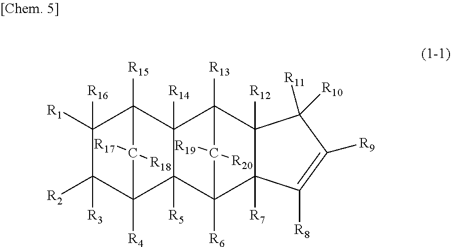

wherein, R₁ to R₂₀ are as shown in Formula (1).

According to one embodiment of the present invention, an alicyclic acrylate-type polymerizable monomer comprising an alicyclic acrylate compound represented by Formula (1-1) (hereinafter, also referred to as “alicyclic acrylate-type polymerizable monomer represented by Formula (1-1)) is provided. Here, the alicyclic acrylate-type polymerizable monomer may be a single alicyclic acrylate compound or a mixture of alicyclic acrylate compounds. The alicyclic acrylate-type polymerizable monomer may be, for example, one or more kinds of alicyclic acrylate compounds, a combination of two or more kinds of alicyclic acrylate compounds, or a combination of two kinds of alicyclic acrylate compounds.

According to a preferred embodiment of the present invention, the alicyclic acrylate-type polymerizable monomer comprising the alicyclic acrylate compound represented by Formula (1-1) comprises a compound (1-1-i) represented by Formula (1-1) where R₁ is a (meth)acryloyloxy group and R₂ is a hydrogen atom, a compound (1-1-ii) represented by Formula (1-1) where R₂ is a (meth)acryloyloxy group and R₁ is a hydrogen atom, or a mixture thereof.

According to a preferred embodiment of the present invention, the alicyclic acrylate-type polymerizable monomer comprising the alicyclic acrylate compound represented by Formula (1-1) comprises a compound in which one of R₁ and R₂ is a methacryloyloxy group and the other of R₁ and R₂ and R₃ to R₂₀ are each a hydrogen atom. A more preferred embodiment of the above is represented by the following Formula (1-1-i-1). Specifically, Formula (1-1-i-1) shows that any one of groups corresponding to R₁ and R₂ bound to the norbornane skeleton in Formula (1) is a methacryloyloxy group.

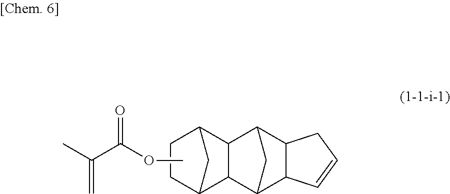

Therefore, the alicyclic acrylate-type polymerizable monomer represented by Formula (1-1-i-1) is a compound represented by the following Formula (1-1-i-2), a compound represented by the following Formula (1-1-i-3), or a mixture thereof (hereinafter, a compound represented by Formula (X) is also referred to as “compound (X)”). The alicyclic acrylate-type polymerizable monomer represented by Formula (1-1-i-1) is preferably a compound represented by Formula (1-1-i-3)

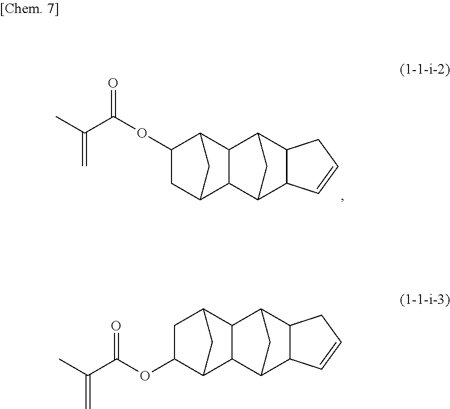 [SMILES: C=C(C)C(=O)OC1CC2CC1C1C3CC(C4C=CCC43)C21 ; C=C(C)C(=O)OC1CC2CC1C1C3CC(C4CC=CC43)C21]

According to one embodiment of the present invention, an alicyclic acrylate compound of the above-described Formula (1) where A is an oxygen atom is an alicyclic epoxy acrylate compound having an epoxy group which is represented by the following Formula (1-2).

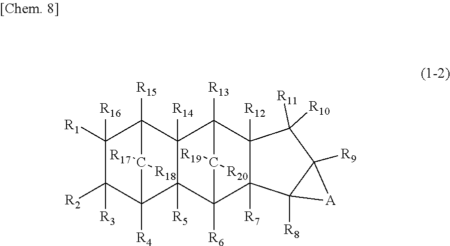

[In the formula, R₁ to R₂₀ are as shown in Formula (1).]

According to one embodiment of the present invention, an alicyclic epoxy acrylate-type polymerizable monomer comprising an alicyclic epoxy acrylate compound represented by Formula (1-2) (hereinafter, also referred to as “alicyclic epoxy acrylate-type polymerizable monomer represented by Formula (1-2)) is provided. Here, the alicyclic epoxy acrylate-type polymerizable monomer may be a single alicyclic epoxy acrylate compound or a mixture of alicyclic epoxy acrylate compounds. The alicyclic epoxy acrylate-type polymerizable monomer may be, for example, one or more kinds of alicyclic epoxy acrylate compounds, a combination of two or more kinds of alicyclic epoxy acrylate compounds, or a combination of two kinds of alicyclic epoxy acrylate compounds.

According to a preferred embodiment of the present invention, the alicyclic epoxy acrylate-type polymerizable monomer comprising the alicyclic epoxy acrylate compound represented by Formula (1-2) comprises a compound (1-2-i) represented by Formula (1-2) where R₁ is a (meth)acryloyloxy group and R₂ is a hydrogen atom, a compound (1-2-ii) represented by Formula (1-2) where R₂ is a (meth)acryloyloxy group and R₁ is a hydrogen atom, or a mixture thereof.

According to a preferred embodiment of the present invention, in the alicyclic epoxy acrylate-type polymerizable monomer comprising the alicyclic epoxy acrylate compound represented by Formula (1-2), one of R₁ and R₂ is a methacryloyloxy group and the other of R₁ and R₂ and R₃ to R₂₀ are each a hydrogen atom. A more preferred embodiment of the above is represented by the following Formula (1-2-i-1). Specifically, Formula (1-2-i-1) shows that any one of groups corresponding to R₁ and R₂ bound to the norbornane skeleton in Formula (1) is a methacryloyloxy group.

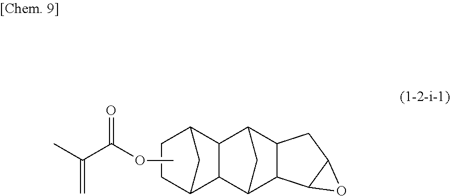

Therefore, the alicyclic epoxy acrylate-type polymerizable monomer represented by Formula (1-2-i-1) is a compound represented by the following Formula (1-2-i-2), a compound represented by the following Formula (1-2-i-3), or a mixture thereof. The alicyclic epoxy acrylate-type polymerizable monomer represented by Formula (1-2-i-1) is preferably a compound represented by Formula (1-2-i-3).

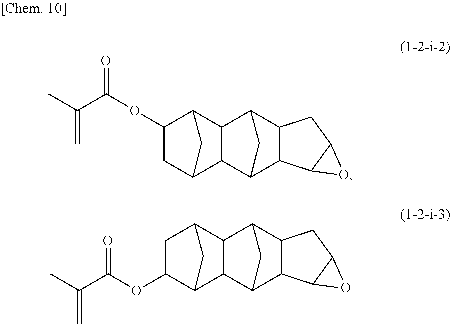

### Method of Producing Alicyclic Acrylate Compound (Compound of Formula (1-1))

The above-described alicyclic acrylate compound represented by Formula (1-1) can be obtained by a method comprising a step of reacting a compound represented by the following Formula (1-a) optionally with an acrylic compound.

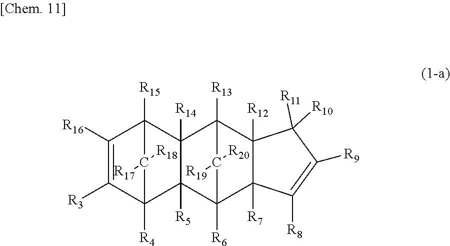

[In the formula, R₃ to R₂₀ are each independently selected from the group consisting of a hydrogen atom, an alkyl group, and an alkoxy group.]

According to one embodiment of the present invention, the method of producing an alicyclic acrylate compound comprises a step of reacting the above-described compound represented by Formula (1-a) with an acrylic compound.

According to one embodiment of the present invention, in the production of an alicyclic acrylate compound, it is preferable to perform purification by distillation, a column, or recrystallization in cases where the purity of an alicyclic acrylate compound obtained by the above-described production method is low.

According to one embodiment of the present invention, examples of an acrylic compound that can be used for the production of an alicyclic acrylate compound include (meth)acrylic acid and preferably methacrylic acid. One kind of acrylic compound or, as necessary, a combination of two or more kinds thereof may be used.

According to one embodiment of the present invention, the amount of the acrylic compound used in the production of the alicyclic acrylate compound is, but is not particularly limited to, preferably from 0.10 to 1.80 mol and more preferably from 0.50 to 1.50 mol with respect to 1.00 mol of the compound represented by Formula (1-a).

Examples of the compound represented by Formula (1-a) include a compound of Formula (1-a-1), which can be obtained by allowing cyclopentadiene and dicyclopentadiene to undergo the Diels-Alder reaction.

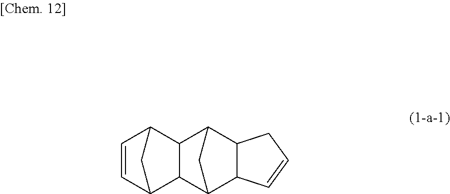 [SMILES: C1=CC2C(C1)C1CC2C2C3C=CC(C3)C12]

According to one embodiment of the present invention, it is preferable to produce an alicyclic acrylate compound in the presence of an acid as appropriate. Examples of an acid that can be used for the production of an alicyclic acrylate compound include various acids such as inorganic acids and organic acids and combinations thereof. Specifically, as inorganic acids, iron (III) chloride, Lewis acid such as boron trifluoride, sulfuric acid, hydrogen chloride, hydrochloric acid, phosphoric acid, heteropolyacids, zeolites, clay minerals, and the like can be exemplified, and as organic acids, trifluoromethanesulfonic acid, methanesulfonic acid, paratoluenesulfonic acid, ion exchange resin, and the like or combinations thereof can be exemplified.

The amount of an acid used is usually from 0.001 to 1 mol, preferably from 0.005 to 0.5 mol, and more preferably from 0.01 to 0.1 mol with respect to 1 mol of the compound represented by Formula (1-a). Here, in cases where the acid is a combination of a plurality of acids, the amount of the acid used means the total thereof.

### Starting Material Production Step (Method of Producing Alicyclic Epoxy Acrylate Compound (Compound of Formula (1-2)))

According to one embodiment of the present invention, the above-described alicyclic epoxy acrylate compound represented by Formula (1-2) can be obtained by a method comprising a step of reacting the above-described alicyclic acrylate compound represented by Formula (1-1) with a peracid.

According to one embodiment of the present invention, in the production of an alicyclic epoxy acrylate compound of Formula (1-2), it is preferable to perform purification by distillation, a column, or recrystallization in cases where the purity of an alicyclic epoxy acrylate compound of Formula (1-2) obtained by the above-described production method is low.

According to one embodiment of the present invention, examples of a peracid that can be used in the production of an alicyclic epoxy acrylate compound of Formula (1-2) include organic peracids such as performic acid, peracetic acid, perbenzoic acid, meta-chloroperbenzoic acid, and trifluoroperacetic acid, and hydrogen peroxide. Of these, meta-chloroperbenzoic acid and hydrogen peroxide are preferred because of their excellent availability and reactivity.

The amount of the peracid used in the production of an alicyclic epoxy acrylate compound of Formula (1-2) is preferably from 0.10 to 2.50 mol and more preferably from 0.50 to 1.80 mol with respect to 1.00 mol of the above-described alicyclic acrylate compound represented by Formula (1-1).

### Usefulness of Alicyclic Epoxy Acrylate Compound

According to one embodiment of the present invention, when allowing the curable composition to contain the alicyclic epoxy acrylate compound, a cured product of the curable composition can have high heat resistance. Therefore, the alicyclic epoxy acrylate compound can be appropriately used in various fields involving various coatings of cans, plastics, papers, woods, and the like, inks, adhesives, sealants, resists, sealings, and the like.

More specifically, the alicyclic epoxy acrylate compound can be appropriately used for 3D modeling materials, acid removers, furniture coatings, decorative coatings, car undercoatings, finish coatings, coatings of beverage cans and other cans, UV curable inks, protective films for optical disc recording layer, color filter protective films, adhesives for bonding optical discs, adhesives for optical materials, die bonding of semiconductor devices, sealing materials for organic EL displays, CCD, sealants for light receiving devices such as infrared sensors, sealants for light emitting devices such as LEDs and organic EL, optical wiring boards, optical connectors, optical semiconductor related parts such as lenses, optical waveguides, photoresists, and composite glass such as tempered glass and security glass. It is also useful as a monomer constituting a polymer and a precursor of a silane coupling agent.

### Epoxy Group-Containing (Meth)Acrylic Polymer

According to one embodiment of the present invention, the epoxy group-containing (meth)acrylic polymer is an epoxy group-containing (meth)acrylic polymer comprising at least a polymerization unit represented by the following Formula (2).

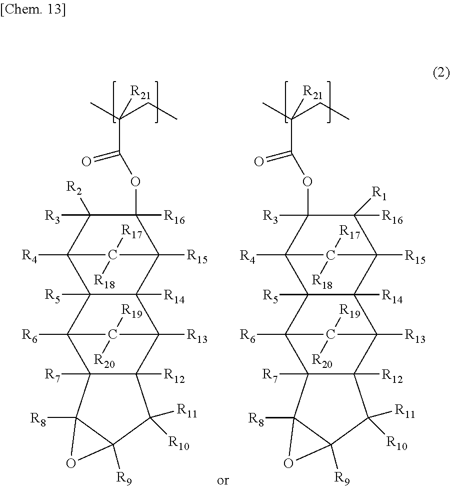

wherein, R₁ and R₂ are each a hydrogen atom,

R₃ to R₂₀ are each independently selected from the group consisting of a hydrogen atom, an alkyl group, and an alkoxy group, and

R₂₁ is a hydrogen atom or a methyl group.

In an epoxy group-containing (meth)acrylic polymer comprising the polymerization unit represented by Formula (2), R₃ to R₂₀ are each independently selected from the group consisting of a hydrogen atom, an alkyl group, and an alkoxy group, preferably selected from a hydrogen atom and an alkyl group, and more preferably a hydrogen atom.

Preferably, R₃ and R₁₆ are each a hydrogen atom in Formula (2).

The number of carbon atoms in alkyl groups of R₃ to R₂₀ in the above-described Formula (2) is, but is not particularly limited to, preferably from 1 to 10 and more preferably from 1 to 5. The above-described alkyl groups may be a linear alkyl group or branched alkyl group.

Examples of the linear alkyl group include a methyl group, an ethyl group, an n-propyl group, and an n-butyl group.

Examples of the branched alkyl group include an isopropyl group, an isobutyl group, and a t-butyl group.

The number of carbon atoms in alkoxy groups of R₃ to R₂₀ in the above-described Formula (2) is, but is not particularly limited to, preferably from 1 to 10 and more preferably from 1 to 5.

The alkyl groups as part of the above-described alkoxy groups may be a linear alkyl group or branched alkyl group.

Examples of an alkoxy group include a methoxy group, an ethoxy group, a propyloxy group, an i-propyloxy group, an n-butoxy group, an i-butoxy group, an s-butoxy group, and a t-butoxy group.

R₂₁ in Formula (2) above is a hydrogen atom or a methyl group and preferably a methyl group.

According to a preferred embodiment of the present invention, the epoxy group-containing (meth)acrylic polymer comprises a polymerization unit represented by the following Formula (2-1), a polymerization unit represented by the following Formula (2-2), or a combination thereof. Therefore, the polymerization unit represented by Formula (2) may be a polymerization unit represented by the following Formula (2-1), a polymerization unit represented by the following Formula (2-2), or a combination thereof. Here, in Formula (2-1) and Formula (2-2) above, R₁ to R₂₁ may be different from one another.

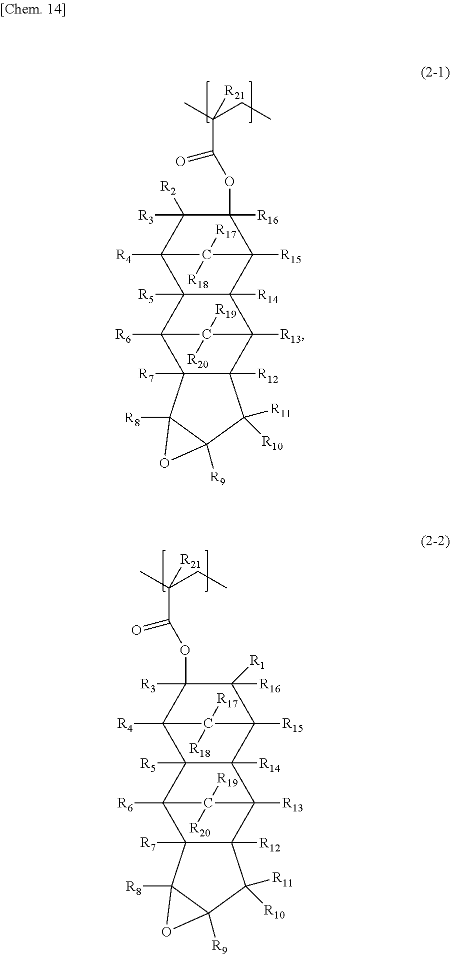

According to a preferred embodiment of the present invention, R₃ to R₂₀ are each a hydrogen atom and R₂₁ is a methyl group in Formula (2) above. A more preferred embodiment of the above is represented by the following Formula (2-3). Note that in Formula (2-3), the position of the bond between the methacryloyloxy group and the norbornane skeleton is not specified, but the methacryloyloxy group-derived oxygen atom is assumed to be bound to the norbornane skeleton at the position corresponding to either R₁ or R₂ in Formula (1).

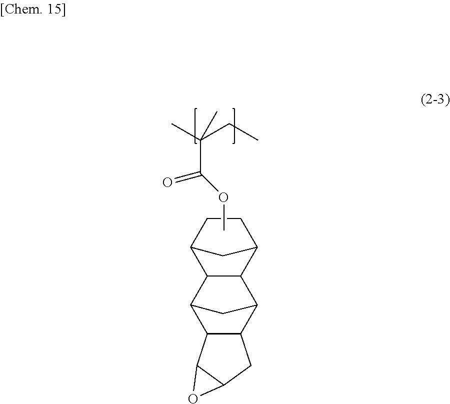

According to one embodiment of the present invention, the polymerization unit represented by Formula (2-3) is a polymerization unit represented by the following Formula (2-4), a polymerization unit represented by the following Formula (2-5), or a combination thereof.

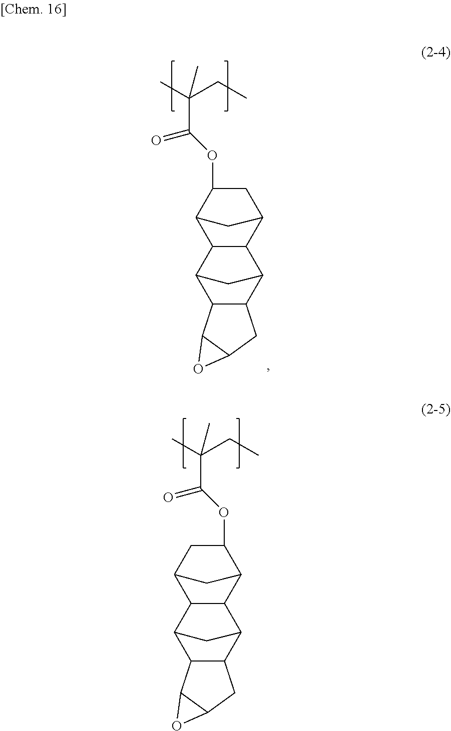

According to one embodiment of the present invention, the epoxy group-containing (meth)acrylic polymer comprises a polymerization unit represented by Formula (2-4), a polymerization unit represented by the following Formula (2-5), or a combination thereof.

According to a more preferred embodiment of the present invention, the above-described polymerization unit represented by Formula (2-3) is the polymerization unit represented by Formula (2-4).

Note that, according to one embodiment of the present invention, the above-described polymerization unit represented by Formula (2) may be a combination of polymerization units in which R₁ to R₂₁ are different from one another. The present invention also encompasses such an aspect.

According to one embodiment of the present invention, the content of the polymerization unit represented by Formula (2) in the epoxy group-containing (meth)acrylic polymer is, for example, 80 mol % or more, preferably 90 mol % or more, and more preferably 95 mol % or more the total amount (100 mol %) of all polymerization units in the epoxy group-containing (meth)acrylic polymer. The upper limit thereof is not particularly limited, but it is, for example, 100 mol % or less.

According to one embodiment of the present invention, the number average molecular weight (Mn) of the epoxy group-containing (meth)acrylic polymer is, for example, 500 or more, and can be preferably from 1,000 to 200,000 and more preferably from 10,000 to 50,000. The number average molecular weight can be measured by size exclusion chromatography.

According to one embodiment of the present invention, the weight average molecular weight (Mw) of the epoxy group-containing (meth)acrylic polymer is, for example, 5,000 or more, and can be preferably from 10,000 to 200,000 and more preferably from 50,000 to 100,000. The weight average molecular weight can be measured by size exclusion chromatography.

### Method of Producing Epoxy Group-Containing (Meth)Acrylic Polymer

According to one embodiment of the present invention, the epoxy group-containing (meth)acrylic polymer can be obtained by a method comprising a step of allowing the above-described alicyclic epoxy acrylate compound of Formula (1-2) to undergo radical polymerization.

According to one embodiment of the present invention, in radical polymerization, a radical polymerization method in a normal acrylic resin can be appropriately selected according to the common technical knowledge well known to those skilled in the art.

According to one embodiment of the present invention, in the method of producing an epoxy group-containing (meth)acrylic polymer, preferably, the epoxy group-containing (meth)acrylic polymer can be obtained by a method comprising a step of allowing the above-described alicyclic epoxy acrylate compound of Formula (1-2) to undergo radical polymerization in the presence of a photoradical polymerization initiator or a thermal radical polymerization initiator. In the above-described step, further, an epoxy compound which is different from the alicyclic epoxy acrylate compound of Formula (1-2) and/or the epoxy group-containing (meth)acrylic polymer comprising the polymerization unit represented by Formula (2) may be additionally mixed. By mixing such an epoxy compound, a mixture of an epoxy group-containing (meth)acrylic polymer and an epoxy compound can be obtained. Specific examples of the photoradical polymerization initiator and the thermal radical polymerization initiator described above include a photoradical polymerization initiator and a thermal radical polymerization initiator which are used for a curable composition described later.

According to one embodiment of the present invention, the amount of a photoradical polymerization initiator or a thermal radical polymerization initiator used in the production of an epoxy group-containing (meth)acrylic polymer is preferably from 0.1 to 100 mol % and more preferably from 1 to 30 mol % with respect to alicyclic epoxy acrylate compound of Formula (1-2).

In the case of using a thermal radical polymerization initiator as the radical polymerization initiator, it is preferable to perform agitation at 50° C. to 150° C. for 1 to 10 hours, for example, in the above-described radical polymerization step.

In the case of using a photoradical polymerization initiator as the radical polymerization initiator, the cumulative irradiation dose (accumulated amount of light) of an active energy ray may be, for example, from 100 to 10000 mJ/cm² and is preferably from 500 to 4000 mJ/cm² in the above-described radical polymerization step. Here, the cumulative irradiation dose (accumulated amount of light) refers to the irradiation dose expressed by the product of irradiation intensity and irradiation time of an active energy ray (e.g., visible ray, UV ray, X-ray, or electron beam)

### Usefulness of Epoxy Group-Containing (Meth)Acrylic Polymer

According to one embodiment of the present invention, when allowing the curable composition to contain the epoxy group-containing (meth)acrylic polymer, a cured product of the curable composition can have high heat resistance. Therefore, it can be used as with the above-described alicyclic epoxy acrylate compound.

### Radical-Polymerizable Compound

According to one embodiment of the present invention, the radical-polymerizable compound is a radical-polymerizable compound comprising at least a polymerization unit represented by the following Formula (3).

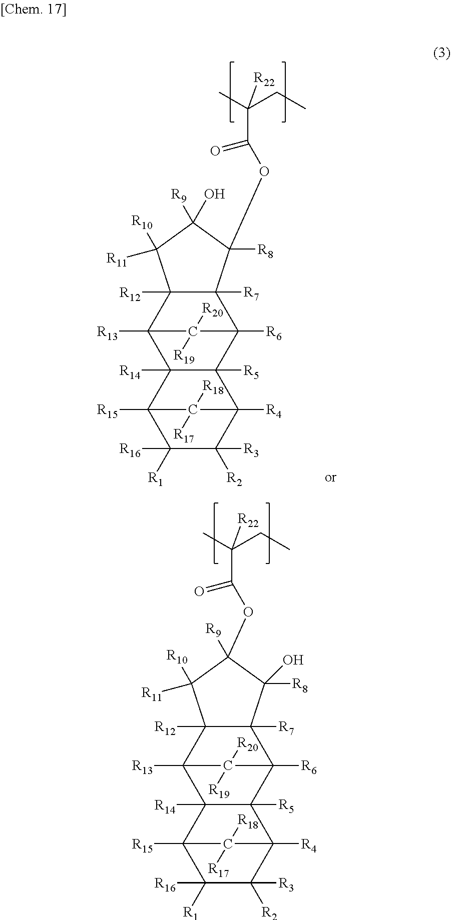

wherein, one of R₁ and R₂ is a (meth)acryloyloxy group,

the other of R₁ and R₂ is a hydrogen atom,

R₃ to R₂₀ are each independently selected from the group consisting of a hydrogen atom, an alkyl group, and an alkoxy group, and

R₂₂ is selected from a hydrogen atom and a methyl group.

In the polymerization unit represented by Formula (3), R₃ to R₂₀ are each independently selected from the group consisting of a hydrogen atom, an alkyl group, and an alkoxy group, preferably selected from the group consisting of a hydrogen atom and an alkyl group, and more preferably a hydrogen atom.

In the polymerization unit represented by Formula (3), preferably, R₃ and R₁₆ are each a hydrogen atom.

The number of carbon atoms in alkyl groups of R₃ to R₂₀ in the above-described Formula (3) is, but is not particularly limited to, preferably from 1 to 10 and more preferably from 1 to 5. The above-described alkyl groups may be a linear alkyl group or branched alkyl group.

Examples of the linear alkyl group include a methyl group, an ethyl group, a propyl group, and an n-butyl group.

Examples of the branched alkyl group include an isopropyl group, an isobutyl group, and a t-butyl group.

The number of carbon atoms in alkoxy groups of R₃ to R₂₀ in the above-described Formula (3) is, but is not particularly limited to, preferably from 1 to 10 and more preferably from 1 to 5.

Examples of an alkoxy group include a methoxy group, an ethoxy group, a propyloxy group, an i-propyloxy group, an n-butoxy group, an i-butoxy group, an s-butoxy group, and a t-butoxy group.

R₂₂ in Formula (3) above is a hydrogen atom or a methyl group and preferably a hydrogen atom.

According to a preferred embodiment of the present invention, the radical-polymerizable compound comprises a polymerization unit represented by the following Formula (3-i), a polymerization unit represented by the following Formula (3-ii), or a combination thereof. Therefore, the polymerization unit represented by Formula (3) may be a polymerization unit represented by the following Formula (3-i), a polymerization unit represented by the following Formula (3-ii), or a combination thereof. Here, in Formula (3-i) and Formula (3-ii) above, R₁ to R₂₀, and R₂₂ may be different from one another.

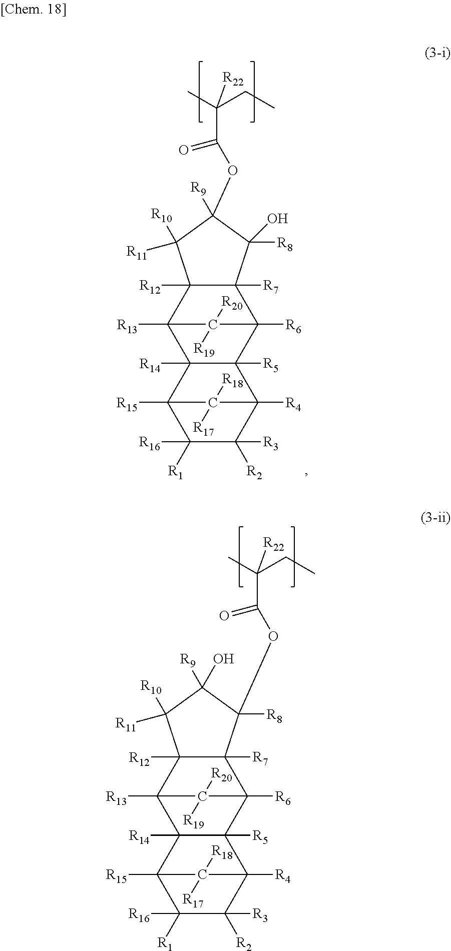

According to a preferred embodiment of the present invention, in Formula (3) above, one of R₁ and R₂ is a methacryloyloxy group and the other of R₁ and R₂, R₃ to R₂₀, and R₂₂ are each a hydrogen atom. A more preferred embodiment of the above is represented by the following Formulas (3-i-1) and (3-ii-1). Specifically, Formulas (3-i-1) and (3-ii-1) show that any one of groups corresponding to R₁ and R₂ bound to the norbornane skeleton in Formula (3) is a methacryloyloxy group.

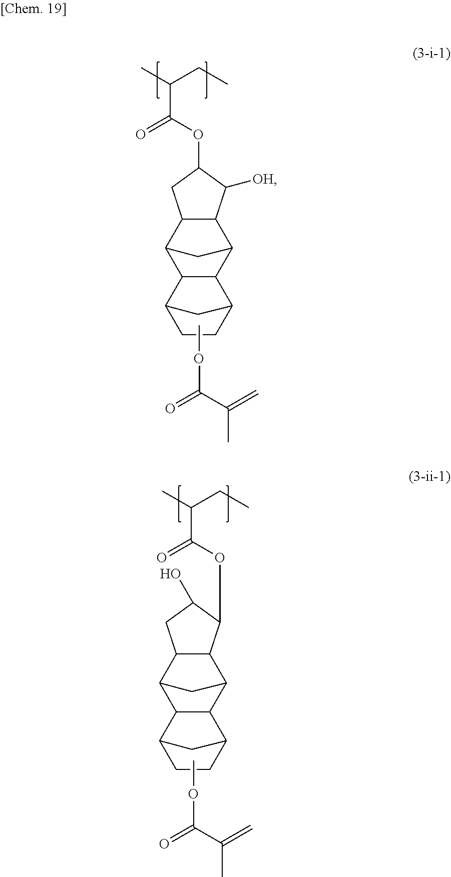

According to one embodiment of the present invention, the polymerization unit represented by Formula (3-i-1) is a polymerization unit represented by the following Formula (3-i-1-1), a polymerization unit represented by the following Formula (3-i-1-2), or a combination thereof. The polymerization unit represented by Formula (3-ii-1) is a polymerization unit represented by the following Formula (3-ii-1-1), a polymerization unit represented by the following Formula (3-ii-1-2), or a combination thereof.

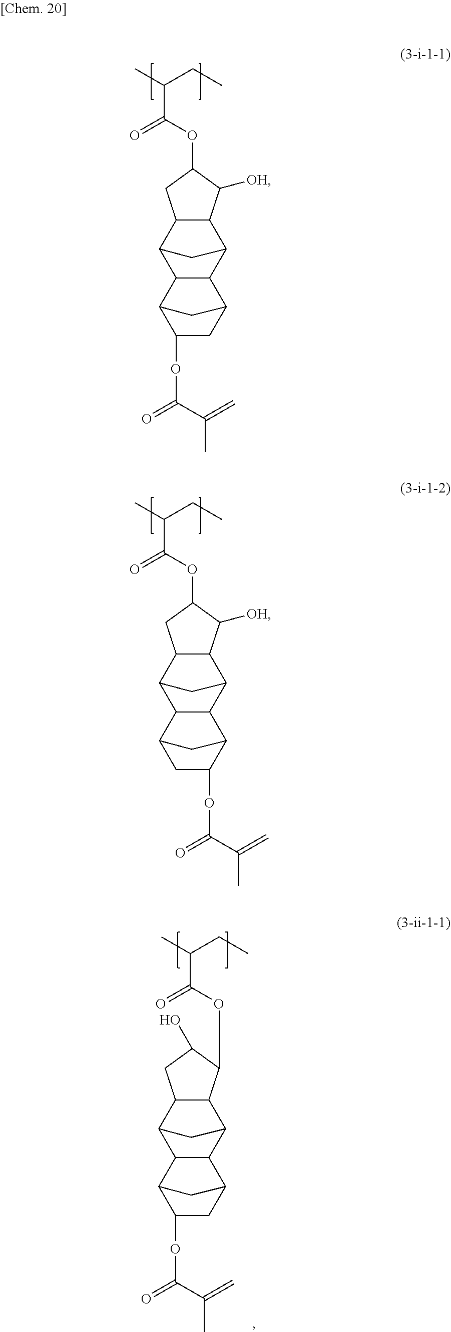

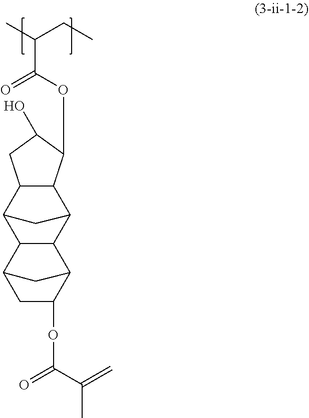

According to one embodiment of the present invention, as the polymerization unit represented by Formula (3-i-1), a combination of the polymerization unit represented by Formula (3-i-1-1) and the polymerization unit represented by (3-i-1-2) can be mentioned.

According to one embodiment of the present invention, the radical-polymerizable compound comprises the polymerization unit represented by Formula (3-i-1-1), the polymerization unit represented by Formula (3-i-1-2), or a combination thereof.

According to one embodiment of the present invention, the radical-polymerizable compound comprises the polymerization unit represented by (3-ii-1-1), the polymerization unit represented by Formula (3-ii-1-2), or a combination thereof.

According to a preferred embodiment of the present invention, the above-described polymerization unit represented by Formula (3-i-1) is the polymerization unit represented by Formula (3-i-1-2).

According to a preferred embodiment of the present invention, the above-described polymerization unit represented by Formula (3-ii-1) is the polymerization unit represented by Formula (3-ii-1-2).

Note that, according to one embodiment of the present invention, the above-described polymerization unit represented by Formula (3) may be a combination of polymerization units in which R₁ to R₂₀ and R₂₂ are different from one another. The present invention also encompasses such an aspect.

According to one embodiment of the present invention, the radical-polymerizable compound further comprises a polymerization unit derived from at least one acrylic compound selected from (meth)acrylic acid ester and (meth)acrylic acid, together with the polymerization unit represented by Formula (3). Examples of such an acrylic compound include (meth)acrylic acid alkyl esters including C₁₋₄ alkyl(meth) acrylates such as (meth)acrylic acid, methyl (meth)acrylate, ethyl (meth)acrylate, and butyl(meth)acrylate, which are (meth)acrylic acid esters, and (meth)acrylic acid. Preferred examples of the polymerization unit include a methyl methacrylate-derived polymerization unit.

Therefore, according to one embodiment of the present invention, the radical-polymerizable compound may further comprise a polymerization unit represented by the following Formula (3-1) together with the polymerization unit represented by Formula (3). Here, the above-described radical-polymerizable compound may comprise a plurality of polymerization units in which R₂₃ and R₂₄ are different.

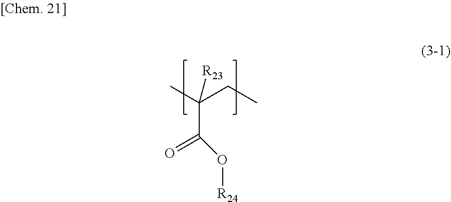

wherein, R₂₃ is selected from a hydrogen atom and a methyl group, and

R₂₄ is selected from a hydrogen atom and an alkyl group.

In the polymerization unit represented by Formula (3-1), R₂₃ is selected from a hydrogen atom or a methyl group and is preferably a methyl group.

The number of carbon atoms in an alkyl group of R₂₄ in the above-described Formula (3-1) is, but is not particularly limited to, preferably from 1 to 10 and more preferably from 1 to 5. The above-described alkyl groups may be a linear alkyl group or branched alkyl group, but is preferably a linear alkyl group.

Examples of the linear alkyl group include a methyl group, an ethyl group, a propyl group, and an n-butyl group, and preferably a methyl group.

Examples of the branched alkyl group include an isopropyl group, an isobutyl group, and a t-butyl group.

In Formula (3-1), it is more preferable that R₂₃ is a methyl group and R₂₄ is a methyl group.

According to one embodiment of the present invention, the content of the polymerization unit represented by Formula (3) in the radical-polymerizable compound can be from 10 to 90 mol %, preferably from 20 to 80 mol %, and more preferably from 30 to 70 mol % with respect to the total amount (100 mol %) of all polymerization units in the radical-polymerizable compound.

According to one embodiment of the present invention, the content of the polymerization unit represented by Formula (3-1) in the radical-polymerizable compound can be from 90 to 10 mol %, preferably from 80 to 20 mol %, and more preferably from 70 to 30 mol % with respect to the total amount (100 mol %) of all polymerization units in the radical-polymerizable compound.

According to another embodiment of the present invention, the butyl(meth)acrylate of the polymerization unit represented by Formula (3) and the polymerization unit represented by Formula (3-1) (the polymerization unit represented by Formula (3): the polymerization unit represented by Formula (3-1)) in the radical-polymerizable compound is preferably from 1:0.05 to 1:10 and more preferably from 1:0.1 to 1:1 on a mass basis. In addition, the content ratio (the polymerization unit represented by Formula (3): the polymerization unit represented by Formula (3-1)) is preferably from 1:0.1 to 1:10, and more preferably from 1:0.5 to 1:1 on a mole basis.

According to one embodiment of the present invention, the number average molecular weight (Mn) of the radical-polymerizable compound is, for example, 500 or more, and it can be preferably from 1,000 to 200,000 and more preferably from 3,000 to 30,000. The number average molecular weight can be measured by size exclusion chromatography.

According to one embodiment of the present invention, the weight average molecular weight (Mw) of the radical-polymerizable compound is, for example, 5,000 or more, and it can be preferably from 3,000 to 200,000 and more preferably from 5,000 to 50,000. The weight average molecular weight can be measured by size exclusion chromatography.

### Method of Producing Radical-Polymerizable Compound

According to one embodiment of the present invention, the radical-polymerizable compound containing the polymerization unit of Formula (3) can be obtained by a method comprising a step (ii) of reacting a polymer (hereinafter, also referred to as “trunk polymer”) of at least one acrylic compound selected from (meth)acrylic acid ester and (meth)acrylic acid with the above-described alicyclic epoxy acrylate compound of Formula (1-2).

According to a preferred embodiment of the present invention, the method of producing the radical-polymerizable compound containing the polymerization unit of Formula (3) may comprise a step (i) of preparing a trunk polymer before the step (ii) of reacting the above-described trunk polymer with the alicyclic epoxy acrylate compound.

### Trunk Polymer Preparation Step

The above-described trunk polymer preparation step is a step of performing radical polymerization of a polymer of at least one acrylic compound selected from (meth)acrylic acid ester and (meth)acrylic acid, and a method of radical polymerization of a normal acrylic resin can be appropriately selected according to the common technical knowledge widely known to those skilled in the art.

According to one embodiment of the present invention, the trunk polymer preparation step may be preferably a step of performing radical polymerization of at least one acrylic compound selected from (meth)acrylic acid ester and (meth)acrylic acid in the presence of a photoradical polymerization initiator or a thermal radical polymerization initiator. Specific examples of the photoradical polymerization initiator and the thermal radical polymerization initiator described above include a photoradical polymerization initiator and a thermal radical polymerization initiator which are used for a curable composition described later.

Examples of the above-described at least one acrylic compound selected from (meth)acrylic acid ester and (meth)acrylic acid include: (meth)acrylic acid alkyl esters including C₁₋₄ alkyl(meth)acrylates such as methyl (meth)acrylate, ethyl (meth)acrylate, and butyl(meth)acrylate, which are (meth)acrylic acid esters, (meth)acrylic acid, or combinations thereof, and preferably methyl (meth)acrylate, (meth)acrylic acid, or a combination thereof, and more preferably a combination of methyl methacrylate and acrylic acid.

Therefore, examples of a polymer (trunk polymer) of at least one acrylic compound selected from (meth)acrylic acid ester and (meth)acrylic acid which is obtained in the trunk polymer preparation step include a (co)polymer of (meth)acrylic acid ester, a (co)polymer of (meth)acrylic acid, and a (co)polymer of (meth)acrylic acid ester and (meth)acrylic acid, and preferably a copolymer of (meth)acrylic acid ester and (meth)acrylic acid and more preferably a copolymer of methacrylic acid ester and acrylic acid. Specific examples of such trunk polymers include (meth)acrylic acid alkyl esters including C₁₋₄ alkyl(meth)acrylates such as methyl (meth)acrylate, ethyl (meth)acrylate, and butyl(meth)acrylate, or polymers of combinations thereof, and preferably a copolymer of methyl (meth)acrylate and (meth)acrylic acid and more preferably a copolymer of methyl methacrylate and acrylic acid.

According to one embodiment of the present invention, the amount of a photoradical polymerization initiator or a thermal radical polymerization initiator used in the trunk polymer preparation step is preferably from 0.1 to 20 mol % and more preferably from 1 to 10 mol % with respect to the acrylic compound.

In the case of using a thermal radical polymerization initiator as the radical polymerization initiator, it is preferable to perform agitation in the presence of a solvent at 50° C. to 150° C. for 1 to 10 hours, for example, in the above-described radical polymerization step.

In the case of using a photoradical polymerization initiator as the radical polymerization initiator, the cumulative irradiation dose (accumulated amount of light) of an active energy ray may be, for example, from 100 to 5000 mJ/cm² and is preferably from 300 to 4000 mJ/cm² in the above-described radical polymerization step. Here, the cumulative irradiation dose (accumulated amount of light) refers to the irradiation dose expressed by the product of irradiation intensity and irradiation time of an active energy ray (e.g., visible ray, UV ray, X-ray, or electron beam)

### Radical-Polymerizable Compound Preparation Step

According to one embodiment of the present invention, the method of producing the radical-polymerizable compound containing the polymerization unit of Formula (3) comprises the step (ii) of reacting a trunk polymer with the above-described alicyclic epoxy acrylate compound of Formula (1-2).

According to one embodiment of the present invention, in the production of the radical-polymerizable compound containing the polymerization unit of Formula (3), it is preferable to perform purification by distillation, a column, or recrystallization in cases where the purity of a radical-polymerizable compound obtained by the above-described method is low.

According to one embodiment of the present invention, the amount of the alicyclic epoxy acrylate compound of Formula (1-2) used in the production of the radical-polymerizable compound is, but is not particularly limited to, preferably from 0.50 to 2.00 mol and more preferably from 0.80 to 1.50 mol with respect to 1.00 mol of carboxylic acid in the trunk polymer.

According to one embodiment of the present invention, it is preferable to perform agitation at 80° C. to 150° C. for 4 to 10 hours, for example, in the above-described radical polymerization step.

According to one embodiment of the present invention, it is preferable to produce the radical-polymerizable compound in the presence of a solvent (e.g., Pph3 or PBu3) and/or a polymerization inhibitor (e.g., hydroquinone monomethylether, hydroquinone, or 4-methoxyphenol) as appropriate.

The amount of the catalyst used is usually from 0.01 to 10 mol %, preferably from 0.1 to 5 mol %, based on 1 mol of the alicyclic epoxy acrylate compound of Formula (1-2).

The amount of the polymerization inhibitor used is usually from 0.01 to 10 mol %, preferably from 0.1 to 5 mol %, based on 1 mol of the alicyclic epoxy acrylate compound of Formula (1-2).

### Usefulness of Radical-Polymerizable Compound

According to one embodiment of the present invention, when allowing the curable composition to contain the radical-polymerizable compound, a cured product of the curable composition can have high heat resistance. Therefore, it can be used as with the above-described alicyclic epoxy acrylate compound.

### Curable Composition

According to the present invention, a curable composition having high heat resistance can be provided by combining an alicyclic acrylate-type polymerizable monomer comprising the above-described alicyclic epoxy acrylate compound represented by Formula (1-2) and an epoxy group-containing (meth)acrylic polymer containing the polymerization unit of Formula (2) or a radical-polymerizable compound containing the polymerization unit of Formula (3) optionally together with other components (e.g., a curing agent, a thermal cationic polymerization initiator, a photo-cationic polymerization initiator, a thermal radical polymerization initiator, an epoxy compound which is different from the above-described alicyclic epoxy acrylate compound and/or epoxy group-containing (meth)acrylic polymer, and a radical-polymerizable compound which is different from the above-described alicyclic epoxy acrylate compound and/or a radical-polymerizable compound containing the polymerization unit of Formula (3)). Hereinafter, the curable composition will be specifically described.

### Curable Composition Comprising Alicyclic Acrylate-Type Polymerizable Monomer Containing Alicyclic Epoxy Acrylate Compound Represented by Formula (1-2)

According to one embodiment of the present invention, the curable composition is characterized in that it comprises at least an alicyclic acrylate-type polymerizable monomer containing the above-described alicyclic epoxy acrylate compound represented by Formula (1-2).

In addition, it is preferable that the above-described curable composition further comprises at least one selected from the group consisting of a curing agent, a thermal cationic polymerization initiator, and a photo-cationic polymerization initiator, and more preferably further comprises at least one selected from the group consisting of a curing agent, a thermal cationic polymerization initiator, and a photo-cationic polymerization initiator.

Further, it is preferable that the above-described curable composition further comprises a thermal radical polymerization initiator.

Still further, it is preferable that the above-described curable composition further comprises an epoxy compound which is different from the above-described alicyclic epoxy acrylate compound.

Yet further, it is preferable that the above-described curable composition further comprises a radical-polymerizable compound which is different from the above-described alicyclic epoxy acrylate compound.

According to one embodiment of the present invention, the proportion of the alicyclic acrylate-type polymerizable monomer comprising the alicyclic epoxy acrylate compound of Formula (1-2) blended in the curable composition is such that the alicyclic epoxy acrylate compound of Formula (1-2) is within the range of from 10 to 80 parts by mass and more preferably from 15 to 65 parts by mass with respect to 100 parts by mass in total of the curable composition. A cured product that is further excellent in heat resistance can be obtained by allowing the alicyclic acrylate-type polymerizable monomer comprising the alicyclic epoxy acrylate compound of Formula (1-2) to be contained in this range.

According to another preferred embodiment of the present invention, the curable composition comprises the alicyclic acrylate-type polymerizable monomer comprising the above-described alicyclic epoxy acrylate compound represented by Formula (1-2) and at least one selected from a curing agent and a thermal cationic polymerization initiator. By allowing the curable composition to comprise the alicyclic acrylate-type polymerizable monomer containing the above-described alicyclic epoxy acrylate compound represented by Formula (1-2), heat resistance of a cured product formed by curing the curable composition can be further improved. Further, it is advantageous in that transparency of a cured product formed by curing the above-described curable composition can be improved.

### Curable Composition Comprising Alicyclic Acrylate-Type Polymerizable Monomer Containing Alicyclic Epoxy Acrylate Compound Represented by Formula (1-2) (Curable Composition 1)

According to another preferred embodiment of the present invention, the curable composition comprises an alicyclic acrylate-type polymerizable monomer comprising the alicyclic epoxy acrylate compound of Formula (1-2), an epoxy compound that is different from the alicyclic epoxy acrylate compound of Formula (1-2), a radical-polymerizable compound that is different from the alicyclic epoxy acrylate compound of Formula (1-2), a thermal cationic polymerization initiator, and a thermal radical polymerization initiator (hereinafter, also referred to as “curable composition 1”). A cured product formed by curing the above-described curable composition can have high heat resistance. Further, it is advantageous in that transparency of a cured product formed by curing the above-described curable composition can be improved.

### Curable Composition Comprising Alicyclic Acrylate-Type Polymerizable Monomer Containing Alicyclic Epoxy Acrylate Compound Represented by Formula (1-2) (Curable Composition 2)

According to another preferred embodiment of the present invention, the curable composition comprises an alicyclic acrylate-type polymerizable monomer comprising the alicyclic epoxy acrylate compound of Formula (1-2), a radical-polymerizable compound that is different from the alicyclic epoxy acrylate compound of Formula (1-2), a curing agent, and a thermal radical polymerization initiator (hereinafter, also referred to as “curable composition 2”). The above-described curable composition may further contain a curing accelerator. A cured product formed by curing the above-described curable composition can have high heat resistance. Further, it is advantageous in that transparency of a cured product formed by curing the above-described curable composition can be improved.

### Curable Composition Comprising Alicyclic Acrylate-Type Polymerizable Monomer Containing Alicyclic Epoxy Acrylate Compound Represented by Formula (1-2) (Curable Composition 3)

According to another preferred embodiment of the present invention, the curable composition comprises an alicyclic acrylate-type polymerizable monomer comprising the alicyclic epoxy acrylate compound of Formula (1-2), an epoxy compound that is different from the alicyclic epoxy acrylate compound of Formula (1-2), and a photo-cationic polymerization initiator (hereinafter, also referred to as “curable composition 3”). The above-described curable composition may further contain a photosensitizer. A cured product formed by curing the above-described curable composition can have high heat resistance.

### Curable Composition Comprising Epoxy Group-Containing (Methylacrylic Polymer Containing Polymerization Unit of Formula (2)

According to one embodiment of the present invention, the curable composition is characterized in that it comprises at least an epoxy group-containing (meth)acrylic polymer containing the above-described polymerization unit of Formula (2).

Further, it is preferable that the above-described curable composition further comprises at least one selected from the group consisting of a curing agent, a thermal cationic polymerization initiator, and a photo-cationic polymerization initiator.

Still further, it is preferable that the above-described curable composition further comprises an epoxy compound that is different from the above-described epoxy group-containing (meth)acrylic polymer.

According to one embodiment of the present invention, the proportion of the epoxy group-containing (meth)acrylic polymer containing the polymerization unit of Formula (2) blended in the curable composition of the present invention is such that the epoxy group-containing (meth)acrylic polymer is within the range of preferably from 5 to 80 parts by mass and more preferably from 15 to 65 parts by mass with respect to 100 parts by mass in total of the curable composition. A cured product that is further excellent in heat resistance can be obtained by allowing the epoxy group-containing (meth)acrylic polymer containing the polymerization unit of Formula (2) to be contained in this range.

### Curable Composition Comprising Epoxy Group-Containing (Meth)Acrylic Polymer Containing Polymerization Unit of Formula (2) (Curable Composition 4)

According to a preferred embodiment of the present invention, the curable composition comprises an epoxy group-containing (meth)acrylic polymer containing the polymerization unit of Formula (2) and a photo-cationic polymerization initiator (hereinafter, also referred to as “curable composition 4”). A cured product formed by curing the above-described curable composition can have high heat resistance.

### Curable Composition Comprising Radical-Polymerizable Compound Containing Polymerization Unit of Formula (3)

According to one embodiment of the present invention, the curable composition is characterized in that it comprises a radical-polymerizable compound containing at least the polymerization unit of Formula (3).

In addition, it is preferable that the above-described curable composition further comprises at least one selected from the group consisting of a curing agent, a thermal cationic polymerization initiator, and a photo-cationic polymerization initiator.

Further, it is preferable that the above-described curable composition comprises a thermal radical polymerization initiator or a photoradical polymerization initiator.

Still further, it is preferable that the above-described curable composition further comprises a radical-polymerizable compound that is different from the radical-polymerizable compound containing the above-described polymerization unit of Formula (3).

According to one embodiment of the present invention, the proportion of the radical-polymerizable compound containing the polymerization unit of Formula (3) blended in the curable composition is such that the radical-polymerizable compound is within the range of from 10 to 90 parts by mass and more preferably from 30 to 80 parts by mass with respect to 100 parts by mass of the curable composition. A cured product that is further excellent in heat resistance can be obtained by allowing the radical-polymerizable compound containing the polymerization unit of Formula (3) to be contained in this range.

### Curable Composition Comprising Radical-Polymerizable Compound Containing Polymerization Unit of Formula (3) (Curable Composition 5)

According to a preferred embodiment of the present invention, the curable composition comprises the radical-polymerizable compound comprising the polymerization unit of Formula (3), a radical-polymerizable compound that is different from the above-described radical-polymerizable compound, and a photoradical polymerization initiator (I) (hereinafter, also referred to as “curable composition 5”). The above-described curable composition may further contain a photosensitizer. A cured product formed by curing the above-described curable composition can have high heat resistance.

Components which can be Contained in Curable Composition Curing Agent

According to one embodiment of the present invention, examples of the curing agent which can be contained in the curable composition include an acid anhydride-based compound, an amine-based compound, a phenol-based compound, and a latent curing agent.

Examples of the acid anhydride-based compound include hexahydrophthalic anhydride, methylhexahydrophthalic anhydride, tetrahydrophthalic anhydride, methyltetrahydrophthalic anhydride, endomethylenetetrahydrophthalic anhydride, methylnadic anhydride, methylbutenyltetrahydrophthalic anhydride, hydrogenated methylnadic anhydride, trialkyltetrahydrophthalic anhydride, cyclohexanetricarboxylic anhydride, methylcyclohexenedicarboxylic anhydride, methylcyclohexanetetracarboxylic acid dianhydride, maleic anhydride, phthalic anhydride, succinic anhydride, dodecenylsuccinic anhydride, octenylsuccinic anhydride, pyromellitic anhydride, trimellitic anhydride, alkylstyrene-maleic anhydride copolymer, chlorendic anhydride, polyazelaic anhydride, benzophenone tetracarboxylic anhydride, ethylene glycol bisanhydrotrimellitate, glycerol tristrimellitate, glycerin bis(anhydrotrimellitate) monoacetate, benzophenonetetracarboxylic acid, polyadipic anhydride, polysebacic anhydride, poly(ethyloctadecanedioic acid) anhydride, poly(phenylhexadecanedioic acid) anhydride, and norbornane-2,3-dicarboxylic anhydride, and preferably hexahydrophthalic anhydride, methylhexahydrophthalic anhydride, or a combination thereof.

Examples of the amine-based compound include polyoxyethylene diamine, polyoxypropylene diamine, polyoxybutylene diamine, polyoxypentylene diamine, polyoxyethylene triamine, polyoxypropylene triamine, polyoxybutylene triamine, polyoxypentylene triamine, diethylene triamine, triethylene tetramine, tetraethylene pentamine, m-xylene diamine, trimethylhexamethylene diamine, 2-methylpentamethylene diamine, diethylaminopropylamine, isophorone diamine, 1,3-bisaminomethylcyclohexane, bis(4-aminocyclohexyl)methane, norbornane diamine, 1,2-diaminocyclohexane, diaminodiphenylmethane, metaphenylene diamine, diaminodiphenyl sulfone, and N-aminoethylpiperazine.

Examples of the phenol-based compound include xylylene skeleton-containing phenol novolac resins, dicyclopentadiene skeleton-containing phenol novolac resins, biphenyl skeleton-containing phenol novolac resins, fluorene skeleton-containing phenol novolac resins, terpene skeleton-containing phenol novolac resins, bisphenol A novolac, bisphenol F novolac, bisphenol S novolac, bisphenol AP novolac, bisphenol C novolac, bisphenol E novolac, bisphenol Z novolac, biphenol novolac, tetramethyl bisphenol A novolac, dimethyl bisphenol A novolac, tetramethyl bisphenol F novolac, dimethyl bisphenol F novolac, tetramethyl bisphenol S novolac, dimethyl bisphenol S novolac, tetramethyl-4,4′-biphenol novolac, trishydroxyphenylmethane novolac, resorcinol novolac, hydroquinone novolac, pyrogallol novolac, diisopropylidene novolac, 1,1-di-4-hydroxyphenylfluorene novolac, phenolated polybutadiene novolac, phenol novolac, cresol novolac, ethylphenol novolac, butylphenol novolac, octylphenol novolac, and naphthol novolac.

Examples of the latent curing agent include dicyandiamide, adipic acid dihydrazide, sebacic acid dihydrazide, dodecanedioic acid dihydrazide, isophthalic acid dihydrazide, ketimines, imidazole compounds, dihydrazide compounds, amine adduct-based latent curing agents. The curable composition may contain one kind, or two or more kinds of the curing agents as described above.

According to a preferred embodiment of the curable composition of the present invention, the curing agent is one or more curing agents selected from the group consisting of acid anhydride-based compounds, amine-based compounds, phenol-based compounds, and latent curing agents, and it is more preferably an acid anhydride-based compound.

According to one embodiment of the present invention, the proportion of the curing agent blended in the curable composition is such that the curing agent is within the range of from 10 to 80 parts by mass and more preferably from 20 to 60 parts by mass with respect to 100 parts by mass in total of the curable composition. A cured product that is further excellent in heat resistance can be obtained by allowing the curing agent to be contained in this range.

### Thermal Cationic Polymerization Initiator

According to one embodiment of the present invention, examples of the thermal cationic polymerization initiator which can be contained in the curable composition include thermal cationic polymerization initiators such as: an onium salt composed of at least one cation selected from aromatic sulfonium, aromatic iodonium, aromatic diazonium, pyridinium, and the like and at least one anion selected from BF₄⁻, PF₆⁻, SbF₆⁻, AsF₆⁻, CF₃SO₃⁻, (CF₃SO₂)₂N⁻, and B(C₆F₅)₄⁻; and an aluminum complex.

Examples of the aromatic sulfonium salt-based thermal cationic polymerization initiator include: hexafluoroantimonate salts such as
(2-ethoxy-1-methyl-2-oxoethyl)methyl-2-naphthalenylsulfonium hexafluoroantimonate,
4-(methoxycarbonyloxy)phenylbenzylmethylsulfonium hexafluoroantimonate, 4-acetoxyphenyldimethylsulfonium hexafluoroantimonate, 4-hydroxyphenylbenzylmethylsulfonium hexafluoroantimonate,
4-hydroxyphenyl(o-methylbenzyl)methylsulfonium hexafluoroantimonate,
4-hydroxyphenyl(α-naphthylmethyl)methylsulfonium hexafluoroantimonate, diphenyl-4-(phenylthio)phenylsulfonium hexafluoroantimonate, triphenylsulfonium hexafluoroantimonate,
bis[4-(di(4-(2-hydroxyethoxy))phenylsulfonio)phenyl]sulfide bishexafluoroantimonate, and
bis[4-(diphenylsulfonio)phenyl]sulfide bishexafluoroantimonate;
hexafluorophosphate salts such as
(2-ethoxy-1-methyl-2-oxoethyl)methyl-2-naphthalenylsulfonium hexafluorophosphate, 4-acetoxyphenylbenzylmethylsulfonium hexafluorophosphate,
4-hydroxyphenyl(o-methylbenzyl)methylsulfonium hexafluorophosphate,
4-hydroxyphenyl(α-naphthylmethyl)methylsulfonium hexafluorophosphate, diphenyl-4-(phenylthio)phenylsulfonium hexafluorophosphate, triphenylsulfonium hexafluorophosphate,
bis[4-(di(4-(2-hydroxyethoxy))phenylsulfonio)phenyl]sulfide bishexafluorophosphate, and
bis[4-(diphenylsulfonio)phenyl]sulfide bishexafluorophosphate;
hexafluoroarsenate salts such as
4-hydroxyphenyl(o-methylbenzyl)methylsulfonium hexafluoroarsenate, and 4-hydroxyphenylbenzylmethylsulfonium hexafluoroarsenate; tetrafluoroborate salts such as
(2-ethoxy-1-methyl-2-oxoethyl)methyl-2-naphthalenylsulfonium tetrafluoroborate,
4-hydroxyphenyl(o-methylbenzyl)methylsulfonium tetrafluoroborate, 4-hydroxyphenylbenzylmethylsulfonium tetrafluoroborate, diphenyl-4-(phenylthio)phenylsulfonium tetrafluoroborate, triphenylsulfonium tetrafluoroborate,
bis[4-(di(4-(2-hydroxyethoxy))phenylsulfonio)phenyl]sulfide bistetrafluoroborate, and bis[4-(diphenylsulfonio)phenyl]sulfide bistetrafluoroborate; trifluoromethanesulfonate salts such as
4-hydroxyphenyl(o-methylbenzyl)methylsulfonium trifluoromethanesulfonate, and
4-hydroxyphenylbenzylmethylsulfonium trifluoromethanesulfonate; trifluoromethanesulfonate salts such as diphenyl-4-(phenylthio)phenylsulfonium trifluoromethanesulfonate; bis(trifluoromethanesulfone)imide salts such as
4-hydroxyphenyl(α-naphthylmethyl)methylsulfonium bis(trifluoromethanesulfone)imide, and
4-hydroxyphenylbenzylmethylsulfonium bis(trifluoromethanesulfone)imide;
tetrakis(pentafluorophenyl)borate salts such as
(2-ethoxy-1-methyl-2-oxoethyl)methyl-2-naphthalenylsulfonium tetrakis(pentafluorophenyl)borate,
4-(methoxycarbonyloxy)phenylbenzylmethylsulfonium tetrakis(pentafluorophenyl)borate,
4-hydroxyphenyl(o-methylbenzyl)methylsulfonium tetrakis(pentafluorophenyl)borate,
4-hydroxyphenyl(α-naphthylmethyl)methylsulfonium tetrakis(pentafluorophenyl)borate,
4-hydroxyphenylbenzylmethylsulfonium tetrakis(pentafluorophenyl)borate,
diphenyl-4-(phenylthio)phenylsulfonium tetrakis(pentafluorophenyl)borate, triphenylsulfonium tetrakis(pentafluorophenyl)borate,
bis[4-(di(4-(2-hydroxyethoxy))phenylsulfonio)phenyl]sulfide tetrakis(pentafluorophenyl)borate, and
bis[4-(diphenylsulfonio)phenyl]sulfide tetrakis(pentafluorophenyl)borate. Monoaryl-based thermal cationic polymerization initiators such as
4-acetoxyphenyldimethylsulfonium hexafluoroantimonate are preferred.

Examples of the aromatic iodonium salt-based thermal cationic polymerization initiator include phenyliodonium hexafluorophosphate, diphenyliodonium hexafluoroantimonate, diphenyliodonium tetrafluoroborate, diphenyliodonium tetrakis(pentafluorophenyl)borate, diphenyliodonium hexafluorophosphate, diphenyliodonium trifluoromethanesulfonate, bis(dodecylphenyl)iodonium hexafluorophosphate, bis(dodecylphenyl)iodonium hexafluoroantimonate, bis(dodecylphenyl)iodonium tetrafluoroborate, bis(dodecylphenyl)iodonium tetrakis(pentafluorophenyl)borate,
4-methylphenyl-4-(1-methylethyl)phenyliodonium hexafluorophosphate,
4-methylphenyl-4-(1-methylethyl)phenyliodonium hexafluoroantimonate,
4-methylphenyl-4-(1-methylethyl)phenyliodonium tetrafluoroborate, and
4-methylphenyl-4-(1-methylethyl)phenyliodonium tetrakis(pentafluorophenyl)borate.

Examples of the aromatic diazonium salt-based thermal cationic polymerization initiator include phenyldiazonium hexafluorophosphate, phenyldiazonium hexafluoroantimonate, phenyldiazonium tetrafluoroborate and phenyldiazonium tetrakis(pentafluorophenyl)borate.

Examples of the pyridinium salt-based thermal cationic polymerization initiator include 1-benzyl-2-cyanopyridinium hexafluorophosphate, 1-benzyl-2-cyanopyridinium hexafluoroantimonate, 1-benzyl-2-cyanopyridinium tetrafluoroborate, 1-benzyl-2-cyanopyridinium tetrakis(pentafluorophenyl)borate,
1-(naphthylmethyl)-2-cyanopyridinium hexafluorophosphate,
1-(naphthylmethyl)-2-cyanopyridinium hexafluoroantimonate,
1-(naphthylmethyl)-2-cyanopyridinium tetrafluoroborate, and
1-(naphthylmethyl)-2-cyanopyridinium tetrakis(pentafluorophenyl)borate.

Examples of the aluminum complex-based thermal cationic polymerization initiator include aluminum carboxylates; aluminum alkoxide, aluminium chloride, aluminum (alkoxide) acetoacetic acid chelate, acetoacetonato aluminum, and ethylacetoacetato aluminum.

Examples of the phosphonium salt-based thermal cationic polymerization initiator include ethyltriphenylphosphonium hexafluoroantimonate, and tetrabutylphosphonium hexafluoroantimonate.

Examples of the quaternary ammonium salt-based thermal cationic polymerization initiator include
N,N-dimethyl-N-benzylanilinium hexafluoroantimonate,
N,N-diethyl-N-benzylanilinium tetrafluoroborate,
N,N-dimethyl-N-benzylpyridinium hexafluoroantimonate,
N,N-diethyl-N-benzylpyridinium trifluoromethanesulfonic acid,
N,N-dimethyl-N-(4-methoxybenzyl)pyridinium hexafluoroantimonate,
N,N-diethyl-N-(4-methoxybenzyl)pyridinium hexafluoroantimonate,
N,N-diethyl-N-(4-methoxybenzyl)toluidinium hexafluoroantimonate, and
N,N-dimethyl-N-(4-methoxybenzyl)toluidinium hexafluoroantimonate.

According to one embodiment of the present invention, the curable composition may contain one kind, or two or more kinds of thermal cationic polymerization initiators as described above.

According to a preferred embodiment of the curable composition of the present invention, the thermal cationic polymerization initiator is preferably selected from the group consisting of aromatic sulfonium salt-based thermal cationic polymerization initiators, aromatic iodonium salt-based thermal cationic polymerization initiators and aluminum complex-based thermal cationic polymerization initiators, and more preferably is an aromatic sulfonium salt-based thermal cationic polymerization initiator.

According to one embodiment of the present invention, the proportion of the thermal cationic polymerization initiator blended in the curable composition is such that the thermal cationic polymerization initiator is within the range of from 0.1 to 20 parts by mass and more preferably from 0.5 to 10 parts by mass with respect to 100 parts by mass in total of the curable composition. A cured product that is further excellent in heat resistance can be obtained by allowing the thermal cationic polymerization initiator to be contained in this range.

### Photo-Cationic Polymerization Initiator

According to one embodiment of the present invention, the photo-cationic polymerization initiator that may be contained in the curable composition is one which generates cationic species or Lewis acid when irradiated with an active energy ray such as a visible ray, UV ray, X-ray, or electron beam, thereby initiating a polymerization reaction of a cationically polymerizable compound. As the photo-cationic polymerization initiator to be contained in the curable composition, it is possible to use, for example, a compound such as an onium salt, a metallocene complex, or an iron-allene complex. Examples of the onium salt which can be used include aromatic sulfonium salts, aromatic diazonium salts, aromatic phosphonium salts and aromatic selenium salts. As the counter ions for these salts, anions such as CF₃SO₃—, BF₄—, PF₆—, AsF₆—, and SbF₆— are used. Among these, it is preferred to use an aromatic sulfonium salt-based photo-cationic polymerization initiator, since it exhibits an excellent curing performance due to having UV absorption properties even in the wavelength range of 300 nm or more, and allows for providing a cured product having a good mechanical strength and adhesion strength. The curable composition may contain two or more kinds of the photo-cationic polymerization initiators.

Examples of the aromatic sulfonium salt include a
diphenyl-4-(phenylthio)phenylsulfonium salt (e.g., diphenyl-4-(phenylthio)phenylsulfonium hexafluorophosphate),
4,4′-bis(diphenylsulfonio)diphenylsulfide bishexafluorophosphate,
4,4′-bis[di(β-hydroxyethoxy)phenylsulfonio]diphenylsulfide bishexafluoroantimonate,
4,4′-bis[di(β-hydroxyethoxy)phenylsulfonio]diphenylsulfide bishexafluorophosphate,
7-[di(p-toluyl)sulfonio]-2-isopropylthioxanthone hexafluoroantimonate,
7-[di(p-toluyl)sulfonio]-2-isopropylthioxanthone tetrakis(pentafluorophenyl)borate,
4-phenylcarbonyl-4′-diphenylsulfonio-diphenylsulfide hexafluorophosphate,
4-(p-tert-butylphenylcarbonyl)-4′-diphenylsulfonio-diphenylsulfide hexafluoroantimonate,
4-(p-tert-butylphenylcarbonyl)-4′-di(p-toluyl)sulfonio-diphenylsulfide tetrakis(pentafluorophenyl)borate,
diphenyl-4-(phenylthio)phenylsulfonium hexafluoroantimonate, triphenylsulfonium trifluoromethanesulfonate,
bis[4-(diphenylsulfonio)phenyl]sulfide bishexafluoroantimonate,
and (4-methoxyphenyl)diphenylsulfonium hexafluoroantimonate.
A diphenyl-4-(phenylthio)phenylsulfonium salt is preferred.

Examples of the aromatic diazonium salt include benzenediazonium hexafluoroantimonate, benzenediazonium hexafluorophosphate, benzenediazonium tetrafluoroborate, and 4-chlorobenzenediazonium hexafluorophosphate.

Examples of the aromatic phosphonium salt include benzyltriphenylphosphonium hexafluoroantimonate.

Examples of the aromatic selenium salt include triphenylselenium hexafluorophosphate.

Examples of the iron-allene complex include xylene-cyclopentadienyl iron (II) hexafluoroantimonate, cumene-cyclopentadienyl iron (II) hexafluorophosphate, and xylene-cyclopentadienyl iron (II) tris(trifluoromethylsulfonyl)methanide.

According to one embodiment of the present invention, the proportion of the photo-cationic polymerization initiator blended in the curable composition is such that the photo-cationic polymerization initiator is within the range of from 0.1 to 20 parts by mass and more preferably from 0.5 to 10 parts by mass with respect to 100 parts by mass in total of the curable composition. A cured product that is further excellent in heat resistance can be obtained by allowing the photo-cationic polymerization initiator to be contained in this range.

### Thermal Radical Polymerization Initiator

1. An alicyclic acrylate compound represented by the following Formula (1): 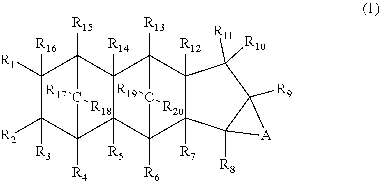
wherein one of R₁ and R₂ is a (meth)acryloyloxy group,
the other of R₁ and R₂ is a hydrogen atom,
R₃ to R₂₀ are each independently selected from the group consisting of a hydrogen atom, an alkyl group, and an alkoxy group, and
A is an oxygen atom or A is not present, and a carbon atom to which R₈ binds and a carbon atom to which R₉ binds together form a double bond.

2. An alicyclic acrylate-type polymerizable monomer comprising the alicyclic acrylate compound according to claim 1.

3. The alicyclic acrylate compound according to claim 1, which is an alicyclic epoxy acrylate compound represented by the Formula (1), wherein A is an oxygen atom.

4. An alicyclic epoxy acrylate-type polymerizable monomer comprising the alicyclic epoxy acrylate compound according to claim 3.

5. A curable composition comprising at least the alicyclic epoxy acrylate-type polymerizable monomer according to claim 4.

6. The curable composition according to claim 5, which further comprises at least one selected from the group consisting of a curing agent, a thermal cationic polymerization initiator, and a photo-cationic polymerization initiator.

7. The curable composition according to claim 6, wherein the curing agent is an acid anhydride-based compound.

8. The curable composition according to claim 5, which further comprises a thermal radical polymerization initiator.

9. The curable composition according to claim 5, which further comprises an epoxy compound that is different from the alicyclic epoxy acrylate compound.

10. The curable composition according to claim 5, which further comprises a radical-polymerizable compound that is different from the alicyclic epoxy acrylate compound.

11. An epoxy group-containing (meth)acrylic polymer comprising at least a polymerization unit represented by the following Formula (2): 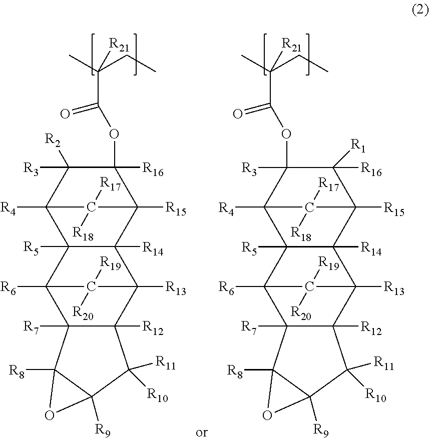
wherein R₁ and R₂ are each a hydrogen atom,
R₃ to R₂₀ are each independently selected from the group consisting of a hydrogen atom, an alkyl group, and an alkoxy group, and
R₂₁ is a hydrogen atom or a methyl group.

12. A curable composition comprising at least the epoxy group-containing (meth)acrylic polymer according to claim 11.

13. The curable composition according to claim 12, which further comprises a photo-cationic polymerization initiator.

14. The curable composition according to claim 12, which further comprises an epoxy compound that is different from the epoxy group-containing (meth)acrylic polymer.

15. A radical-polymerizable compound comprising at least a polymerization unit represented by the following Formula (3): 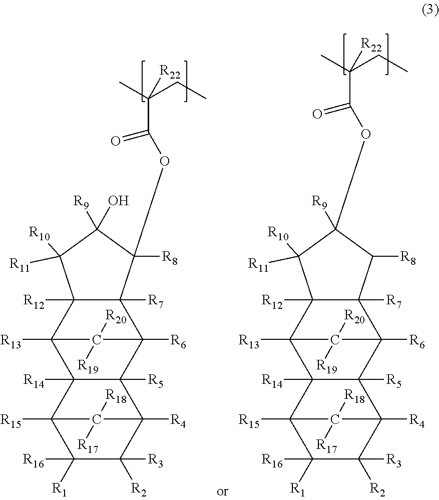
wherein one of R₁ and R₂ is a (meth)acryloyloxy group,
the other of R₁ and R₂ is a hydrogen atom,
R₃ to R₂₀ are each independently selected from the group consisting of a hydrogen atom, an alkyl group, and an alkoxy group, and
R₂₂ is selected from a hydrogen atom and a methyl group.

16. The radical-polymerizable compound according to claim 15, which further comprises a polymerization unit derived from at least one acrylic compound selected from (meth)acrylic acid ester and (meth)acrylic acid.

17. A curable composition comprising at least the radical-polymerizable compound according to claim 15.

18. The curable composition according to claim 17, which further comprises a photoradical polymerization initiator.

19. The curable composition according to claim 17, which further comprises a radical-polymerizable compound that is different from the radical-polymerizable compound.

20. A method of producing an epoxy group-containing acrylic polymer, comprising a step of radically polymerizing the alicyclic epoxy acrylate compound according to claim 3.

21. A method of producing a radical-polymerizable compound, comprising a step of reacting a polymer of at least one acrylic compound selected from (meth)acrylic acid ester and (meth)acrylic acid with the alicyclic epoxy acrylate compound according to claim 3.
